(19) United States
(12) Patent Application Publication
(10) Pub. No.: US 2023/0233614 A1
(43) Pub. Date: Jul. 27, 2023

(54) REGENERATIVE CELL THERAPY FOR VIRAL INDUCED SEXUAL DYSFUNCTION

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Timothy Warbington, Phoenix, AZ (US)

(73) Assignee: CREATIVE MEDICAL TECHNOLOGIES, INC., Phoenix, AZ (US)

(21) Appl. No.: 18/082,907

(22) Filed: Dec. 16, 2022

Related U.S. Application Data
(60) Provisional application No. 63/302,228, filed on Jan. 24, 2022.

(51) Int. Cl. A61K 35/28

(52) U.S. Cl. CPC A61K 35/28

(57) ABSTRACT

The invention provides means, methods, and compositions of matter useful for treatment of viral induced sexual dysfunction. In one embodiment the invention teaches the use of autologous bone marrow mononuclear cells as a source of endothelial repair in penile or clitoral tissues that has been damaged by viral causes. In one embodiment, said viral cause is COVID-19 infection. In some embodiments the invention discloses means of maintaining and/or increasing sexual function, in some cases the invention describes preservation of tissue mass and/or size by administration of regenerative cells. Said cells may be autologous, allogeneic or xenogeneic. In some embodiments the invention teaches the utilization of derivatives of regenerative cells such as exosomes and/or conditioned media.

### CROSS-REFERENCE TO RELATED APPLICATIONS

This application claims the benefit of priority to U.S. Provisional Application No. 63/302,228, filed Jan. 24, 2022, which is incorporated herein by reference in its entirety.

### FIELD OF THE INVENTION

The teachings herein relate to regenerative cell therapy for treating COVID-19 induced sexual dysfunction.

### BACKGROUND OF THE INVENTION

The novel coronavirus (COVID-19) epidemic has caused innumerable damage to many aspects of human life. One particularly intriguing area of COVID-19 pathology is induction of sexual dysfunction. The initial suggestion of COVID-19 associated erectile dysfunction (ED) was came from a paper by Sonsome et al. who hypothesized based on known properties of COVID-19 such as hyperinflammation resulting in a “cytokine storm”, which leads to severe complications, such as the development of micro-thrombosis and disseminated intravascular coagulation (DIC) [1]. Subsequently, numerous reports of COVID-19 associated ED have been described [2-5].

Mechanistically, Kresch et al. showed viral infection of penile tissue. They collected penile tissue from patients undergoing surgery for penile prosthesis for severe ED. Specimens were obtained from two men with a history of COVID-19 infection and two men with no history of infection. Specimens were imaged with TEM and H&E staining. RT-PCR was performed from corpus cavernosum biopsies. The tissues collected were analyzed for endothelial Nitric Oxide Synthase (eNOS, a marker of endothelial function) and COVID-19 spike-protein expression. Endothelial progenitor cell (EPC) function was assessed from blood samples collected from COVID-19 (+) and COVID-19 (−) men. TEM showed extracellular viral particles ˜100 nm in diameter with peplomers (spikes) near penile vascular endothelial cells of the COVID-19 (+) patients and absence of viral particles in controls. PCR showed presence of viral RNA in COVID-19 (+) specimens. eNOS expression in the corpus cavernosum of COVID-19 (+) men was decreased compared to COVID-19 (−) men. Mean EPC levels from the COVID-19 (+) patients were substantially lower compared to mean EPCs from men with severe ED and no history of COVID-19. This demonstrated specific infection and destruction of endothelial cells [6]. The deterioration of endothelial function by COVID-19 and associated with ED was reported by independent groups subsequently [7, 8].

SARS-CoV-2 is an enveloped, positive-sense, single-stranded RNA virus of the subgenus Sarbecovirus which belongs to the genus Betacoronavirus [9, 10]. The main strains of this family are 229E (alpha coronavirus), NL63 (alpha coronavirus), 0C43 (beta coronavirus), and HKU1 (beta coronavirus), which are relatively innocuous and cause the common cold, as well as more virulent strains such as MERS-CoV (the beta coronavirus that causes Middle East Respiratory Syndrome, or MERS) [11-18]. Rapidly after its identification, scientists found that SARS-CoV-2 possesses 88% identity to two bat-derived severe acute respiratory syndrome (SARS)-like coronaviruses, bat-SL-CoVZC45 and bat-SL-CoVZXC21 which were collected in 2018 in Zhoushan, eastern China. It was also found that SARS-CoV-2 has 79% homology to SARS-CoV and 50% homology to MERS-CoV [19].

Pathology of COVID-19 is associated with pulmonary damage caused by localized leukocytic infiltration resulting in lung fluid leakage and reduced respiratory capacity. Negative association between neutrophil infiltration, inflammatory markers in blood, and in BAL exists with patient survival [20]. Suppression of inflammation using various means such as antibodies to IL-6 have shown clinical benefit in some situations [21-23]. Interestingly, IL-6 has been associated with creation of ED in COVID-19 patients. On study that was reported assessed 80 male patients aged 30-45 years who were hospitalised due to COVID-19. The International Index of Erectile Function (IIEF-5) questionnaire was used to assess erectile function. The IIEF-5 questionnaire was re-administered at a 3-month control visit after discharge, and the change score from baseline was recorded. The patients were divided into three groups according to the IIEF-5 score at 3 months as Group 1 (severe ED), Group 2 (moderate ED) and Group 3 (no ED), and into two groups according to IL-6 level at the time of admission as Group A (IL-6<50 ng/ml) and Group B (IL-6 >50 ng/ml). The change in the IIEF-5 score was significantly greater in Group B than in Group A. There was also significant difference in IL-6 between Group 1 and Group 2. The correlation analysis revealed a moderate correlation between IL-6 level and the change in IIEF-5 score and D-dimer level and a weak correlation between IL-6 level and FSH. The present study suggests that elevated IL-6 levels in male patients hospitalised due to COVID-19 might be related to the risk of developing ED [24].

The current treatments for erectile dysfunction include psychotherapy, oral or injected medication, and penile prosthesis. Oral medication is the most popular of these modalities. Presently, many patients seek non- or minimally invasive, permanent treatments, as opposed to temporary improvement. Erectile dysfunction (ED) is characterized by the lack of ability to achieve and maintain penile erection for intercourse. Methods used to quantify ED include the Erectile Function Visual Analog Scale (EF-VAS) and the International Index of Erectile Function (IIEF) [25, 26], however clinically it is primarily diagnosed based on symptomology. In our aging society ED is becoming an increasing problem. According to one study 39% of men at age40 experience some type of ED, whereas at age 70 the incidence rises to 67% [27]. It is estimated that 10-30 million Americans suffer from this condition [28]. In addition to aging it is believed that 50-85% of ED cases are associated with conditions that affect the endothelium such as hypertension, diabetes, cardiovascular disease, and dyslipidemia [29]. The prevalence of ED is illustrated by the fact that just in 2006 in the US over 39 million prescriptions have been written for one ED drug, Viagra [30].

World-wide it is believed that 100 million men are affected by various degrees of ED. Currently ED is treated by oral inhibitors of phosphodiesterase-5 (sildenafil [Viagra, Revatio], tadalafil [Cialis] and vardenafil [Levitra]), which are considered the standard of care for first-line treatment. Unfortunately, 30-40% of patients are unresponsive to therapy or do not tolerate adverse effects associated with treatment [31-33]. In addition, PDE5 inhibitors are known to possess a variety of systemic effects in numerous organ systems, therefore the long term effects of PDE5 inhibition are still uncertain. It is known that PDE5 inhibitors can induce a variety of adverse effects such as optic neuropathy [34], headaches [35], and various cardiovascular pathologies [36], especially when taken in combination with nitrates [37]. In fact, in 1998, the US Food and Drug Administration published a report on 130 confirmed deaths among men who received prescriptions for sildenafil citrate, where causes of death included arrythmias, sudden cardiac death and hypotension-associated events [38]. Beneficial non-ED uses of PDE5 inhibitors are also known, for example, since PDE5 is expressed in lung tissue, investigators sought to, and succeeded at inhibiting symptomatic pulmonary arterial hypertension in a double blind clinical trial [39] by administration of sildenafil citrate. However, given the various areas in the body that PDE5 is expressed, such as platelets, kidneys, and pancreas [40], it is the belief of some that systemic inhibition of this enzymatic system may have adverse physiologic consequences in the long-run [41].

It is becoming increasingly recognized that ED is a symptom of cardiovascular disease, primarily endothelial dysfunction. Several studies suggest the ED is actually one of the first signs of impending cardiovascular disease [42-44]. Therefore one method of treating the cause as opposed to the symptoms is to attempt to heal the endothelium. Before elaborating on these strategies, we will first review the basic biology of erections.

Erectile responses require a coordinated increase in arterial inflow, which originates from the pudendal arteries, relaxation of the corporal smooth muscle, and inhibition of venous outflow [45, 46]. Key to this response is production of nitric oxide (NO) from endothelial cells and nonadrenergic noncholinergic (NANC) postganglionic parasympathetic neurons, as well as responsiveness to this. NO binds to, and activates, the enzyme guanylate cyclase, which in turn catalyzes the generation of cGMP from GTP. As a result, cGMP induces a cascade of signals in the smooth muscle cells resulting in relaxation [47]. Breakdown of cGMP in the cavernosal tissue is mediated by PDE-5. Increasing the duration of NO signaling by preventing cGMP breakdown is the main mechanism of action for the successful PDE-5 inhibitor class of drugs which currently are used as first-line treatment of ED [48]. Interestingly, recent studies have shown that these drugs have other beneficial effects such as stimulation of bone marrow endothelial progenitor cell function [49-53], inhibition of smooth muscle cell apoptosis [54, 55], preservation/restoration of function in post-prostatectomy settings [56, 57] and activation of mesolimbic dopaminergic neurons in the CNS to promote sexual behavior [58].

Currently there are no reproducible means of reducing or treating COVID-19 associated ED. The current invention aims to overcome the current limitations in the art.

### SUMMARY

Preferred embodiments include methods of treating viral induced sexual dysfunction comprising administering into cavernosal or clitoral tissue a regenerative cell population capable of stimulating one or more of the following: a) reduction of endothelial and/or smooth muscle apoptosis and/or neural cell apoptosis; b) stimulation angiogenesis; c) stimulation proliferation of smooth muscle; d) reducing fibrosis; e) augmentation of neurogenesis and f) suppression of inflammation.

Preferred methods include embodiments wherein said regenerative cell population is substituted with derivatives of said regenerative cell population.

Preferred methods include embodiments wherein said derivative of said regenerative cell population is conditioned media.

Preferred methods include embodiments wherein said derivative of said regenerative cell population is microvesicles.

Preferred methods include embodiments wherein said derivative of said regenerative cell population is exosomes.

Preferred methods include embodiments wherein said regenerative cell population is autologous to the patient.

Preferred methods include embodiments wherein said regenerative cell population is allogeneic to the patient.

Preferred methods include embodiments wherein said regenerative cell population is xenogeneic to the patient.

Preferred methods include embodiments wherein said regenerative cell is a mesenchymal stem cell.

Preferred methods include embodiments wherein said sexual dysfunction is caused by a single stranded RNA virus.

Preferred methods include embodiments wherein said sexual dysfunction is caused by a double stranded RNA virus.

Preferred methods include embodiments wherein said sexual dysfunction is caused by a DNA virus.

Preferred methods include embodiments wherein said sexual dysfunction is caused by a coronavirus.

Preferred methods include embodiments wherein said sexual dysfunction is caused by COVID-19.

Preferred methods include embodiments wherein said sexual dysfunction is caused by inflammation associated with viral infection.

Preferred methods include embodiments wherein said inflammation is associated with macrophage activation.

Preferred methods include embodiments wherein said macrophage activation is associated with enhanced production of nitric oxide as compared to a macrophage in a resting state.

Preferred methods include embodiments wherein said macrophage activation is associated with enhanced production of TNF-alpha as compared to a macrophage in a resting state.

Preferred methods include embodiments wherein said macrophage activation is associated with enhanced production of interferon-alpha as compared to a macrophage in a resting state.

Preferred methods include embodiments wherein said macrophage activation is associated with enhanced production of interferon-beta as compared to a macrophage in a resting state.

Preferred methods include embodiments wherein said macrophage activation is associated with enhanced production of interferon-gamma as compared to a macrophage in a resting state.

Preferred methods include embodiments wherein said macrophage activation is associated with enhanced production of interferon-tau as compared to a macrophage in a resting state.

Preferred methods include embodiments wherein said macrophage activation is associated with enhanced production of interferon-omega as compared to a macrophage in a resting state.

Preferred methods include embodiments wherein said macrophage activation is associated with enhanced production of lymphotoxin as compared to a macrophage in a resting state.

The method of c6laim 16, wherein said macrophage activation is associated with enhanced production of PDGF-BB as compared to a macrophage in a resting state.

Preferred methods include embodiments wherein said macrophage activation is associated with enhanced production of interleukin-1 beta as compared to a macrophage in a resting state.

Preferred methods include embodiments wherein said macrophage activation is associated with enhanced production of interleukin-6 as compared to a macrophage in a resting state.

Preferred methods include embodiments wherein said macrophage activation is associated with enhanced production of interleukin-8 as compared to a macrophage in a resting state.

Preferred methods include embodiments wherein said macrophage activation is associated with enhanced production of interleukin-9 as compared to a macrophage in a resting state.

Preferred methods include embodiments wherein said macrophage activation is associated with enhanced production of interleukin-11 as compared to a macrophage in a resting state.

Preferred methods include embodiments wherein said macrophage activation is associated with enhanced production of interleukin-15 as compared to a macrophage in a resting state.

Preferred methods include embodiments wherein said macrophage activation is associated with enhanced production of interleukin-17 as compared to a macrophage in a resting state.

Preferred methods include embodiments wherein said macrophage activation is associated with enhanced production of interleukin-18 as compared to a macrophage in a resting state.

Preferred methods include embodiments wherein said macrophage activation is associated with enhanced production of interleukin-21 as compared to a macrophage in a resting state.

Preferred methods include embodiments wherein said macrophage activation is associated with enhanced production of interleukin-22 as compared to a macrophage in a resting state.

Preferred methods include embodiments wherein said macrophage activation is associated with enhanced production of interleukin-23 as compared to a macrophage in a resting state.

Preferred methods include embodiments wherein said macrophage activation is associated with enhanced production of interleukin-27 as compared to a macrophage in a resting state.

Preferred methods include embodiments wherein said macrophage activation is associated with enhanced production of interleukin-33 as compared to a macrophage in a resting state.

37. Preferred methods include embodiments wherein said macrophage activation is associated with enhanced production of interleukin-37 as compared to a macrophage in a resting state.

Preferred methods include embodiments wherein said inflammation is associated with increased activation of NF-kappa B in cells comprising caveronsal tissue.

Preferred methods include embodiments wherein said cells comprising said cavernosal tissue are endothelial cells.

Preferred methods include embodiments wherein said cells comprising said cavernosal tissue are monocytes.

Preferred methods include embodiments wherein said cells comprising said cavernosal tissue are stems.

Preferred methods include embodiments wherein said cells comprising said cavernosal tissue are smooth muscle cells.

Preferred methods include embodiments wherein said cells comprising said cavernosal tissue are epithelial cells.

Preferred methods include embodiments wherein said cells comprising said cavernosal tissue are telocytes.

Preferred methods include embodiments wherein said cells comprising said cavernosal tissue are neurons.

Preferred methods include embodiments wherein said cells comprising said cavernosal tissue are schwann cells.

Preferred methods include embodiments wherein said inflammation is associated with increased activation of NF-kappa B in cells comprising clitoral tissue.

Preferred methods include embodiments wherein said cells comprising said clitoral tissue are endothelial cells.

Preferred methods include embodiments wherein said cells comprising said clitoral tissue are monocytes.

Preferred methods include embodiments wherein said cells comprising said clitoral tissue are stems.

Preferred methods include embodiments wherein said cells comprising said clitoral tissue are smooth muscle cells.

Preferred methods include embodiments wherein said cells comprising said clitoral tissue are epithelial cells.

Preferred methods include embodiments wherein said cells comprising said clitoral tissue are telocytes.

Preferred methods include embodiments wherein said cells comprising said clitoral tissue are neurons.

Preferred methods include embodiments wherein said cells comprising said clitoral tissue are schwann cells.

Preferred methods include embodiments wherein said inflammation is associated with degradation of the inhibitor of Kappa B (IKK) cells comprising caveronsal tissue.

Preferred methods include embodiments wherein said cells comprising said cavernosal tissue are endothelial cells.

Preferred methods include embodiments wherein said cells comprising said cavernosal tissue are monocytes.

Preferred methods include embodiments wherein said cells comprising said cavernosal tissue are stems.

Preferred methods include embodiments wherein said cells comprising said cavernosal tissue are smooth muscle cells.

Preferred methods include embodiments wherein said cells comprising said cavernosal tissue are epithelial cells.

Preferred methods include embodiments wherein said cells comprising said cavernosal tissue are telocytes.

Preferred methods include embodiments wherein said cells comprising said cavernosal tissue are neurons.

Preferred methods include embodiments wherein said cells comprising said cavernosal tissue are schwann cells.

Preferred methods include embodiments wherein said inflammation is associated with degradation of the inhibitor of kappa B (IKK) in cells comprising clitoral tissue.

Preferred methods include embodiments wherein said cells comprising said clitoral tissue are endothelial cells.

Preferred methods include embodiments wherein said cells comprising said clitoral tissue are monocytes.

Preferred methods include embodiments wherein said cells comprising said clitoral tissue are stems.

Preferred methods include embodiments wherein said cells comprising said clitoral tissue are smooth muscle cells.

Preferred methods include embodiments wherein said cells comprising said clitoral tissue are epithelial cells.

Preferred methods include embodiments wherein said cells comprising said clitoral tissue are telocytes.

Preferred methods include embodiments wherein said cells comprising said clitoral tissue are neurons.

Preferred methods include embodiments wherein said cells comprising said clitoral tissue are schwann cells.

Preferred methods include embodiments wherein said sexual dysfunction is associated with reduced production of interleukin-10 in tissue associated with sexual function as compared to an age-matched control.

Preferred methods include embodiments wherein said sexual dysfunction is associated with reduced production of interleukin-4 in tissue associated with sexual function as compared to an age-matched control.

Preferred methods include embodiments wherein said sexual dysfunction is associated with reduced production of interleukin-1 receptor antagonist in tissue associated with sexual function as compared to an age-matched control.

Preferred methods include embodiments wherein said sexual dysfunction is associated with reduced production of TGF-beta in tissue associated with sexual function as compared to an age-matched control.

Preferred methods include embodiments wherein said sexual dysfunction is associated with reduced production of interleukin=13 in tissue associated with sexual function as compared to an age-matched control.

Preferred methods include embodiments wherein said sexual dysfunction is associated with reduced production of VEGF in tissue associated with sexual function as compared to an age-matched control.

Preferred methods include embodiments wherein said sexual dysfunction is associated with reduced production of HGF in tissue associated with sexual function as compared to an age-matched control.

Preferred methods include embodiments wherein said sexual dysfunction is associated with reduced production of IGF in tissue associated with sexual function as compared to an age-matched control.

Preferred methods include embodiments wherein said sexual dysfunction is associated with reduced production of FGF-1 in tissue associated with sexual function as compared to an age-matched control.

Preferred methods include embodiments wherein said sexual dysfunction is associated with reduced production of FGF-2 in tissue associated with sexual function as compared to an age-matched control.

Preferred methods include embodiments wherein said regenerative cell is a stem cell.

Preferred methods include embodiments wherein said stem cell is a hematopoietic stem cell.

Preferred methods include embodiments wherein said hematopoietic stem cell is capable of generating leukocytic, lymphocytic, thrombocytic and erythrocytic cells when transplanted into an immunodeficient animal.

Preferred methods include embodiments wherein said hematopoietic stem cell is non-adherent to plastic.

Preferred methods include embodiments wherein said hematopoietic stem cell is adherent to plastic.

Preferred methods include embodiments wherein said hematopoietic stem cell is exposed to hyperthermia.

Preferred methods include embodiments wherein said hematopoietic stem cell expresses interleukin-3 receptor.

Preferred methods include embodiments wherein said hematopoietic stem cell expresses interleukin-1 receptor.

Preferred methods include embodiments wherein said hematopoietic stem cell expresses c-met.

Preferred methods include embodiments wherein said hematopoietic stem cell expresses mp1.

Preferred methods include embodiments wherein said hematopoietic stem cell expresses interleukin-11 receptor.

Preferred methods include embodiments wherein said hematopoietic stem cell expresses G-CSF receptor.

Preferred methods include embodiments wherein said hematopoietic stem cell expresses GM-CSF receptor.

Preferred methods include embodiments wherein said hematopoietic stem cell expresses M-CSF receptor.

Preferred methods include embodiments wherein said hematopoietic stem cell expresses VEGF-receptor.

Preferred methods include embodiments wherein said hematopoietic stem cell expresses c-kit.

Preferred methods include embodiments wherein said hematopoietic stem cell expresses CD33.

Preferred methods include embodiments wherein said hematopoietic stem cell expresses CD133.

Preferred methods include embodiments wherein said hematopoietic stem cell expresses CD34.

Preferred methods include embodiments wherein said hematopoietic stem cell expresses Fas ligand.

Preferred methods include embodiments wherein said hematopoietic stem cell does not express lineage markers.

Preferred methods include embodiments wherein said hematopoietic stem cell does not express CD14.

Preferred methods include embodiments wherein said hematopoietic stem cell does not express CD16.

Preferred methods include embodiments wherein said hematopoietic stem cell does not express CD3.

Preferred methods include embodiments wherein said hematopoietic stem cell does not express CD56.

Preferred methods include embodiments wherein said hematopoietic stem cell does not express CD38.

Preferred methods include embodiments wherein said hematopoietic stem cell does not express CD30.

Preferred methods include embodiments wherein said regenerative cell is a mesenchymal stem cell.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue stems, corneal keratocytes, tendon stems, bone marrow reticular tissue stems, nonepithelial stems, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45;

Preferred methods include embodiments wherein said mesenchymal stem cells from umbilical cord tissue express, relative to a human stem, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

### DETAILED DESCRIPTION OF THE INVENTION

The invention provides regeneration of tissue associated with sexual function that has been affected in a detrimental manner by viral infection. In one particular embodiment the invention provides treatment of COVID-19 associated sexual dysfunction through regeneration of cavernosal or clitoral tissue.

Viral infections have previously been shown to alter endothelial cells and endothelial cell activity. For example, numerous viruses induce endothelial activation, as measured by various parameters such as induction of procoagulant activity. Viruses such as African Swine Fever Virus [59-62], cytomegalovirus [63-77], sendai virus [78], herpes simplex virus [79-81], avian hemangioma retrovirus [82], hantavirus [83-88], dengue virus [89-94], HIV [95-113], Lassa Fever [114], Semliki Forest virus [115], vesicular stomatitis virus [116], influenza virus [117-119], hepatitis C [120-122], Junin virus [123, 124], Cocksackie B virus [125, 126], Epstein-Barr virus [127], kaposi's sarcoma associated herpes virus [128, 129], human parvovirus B19 [130], Bluetongue virus [131].

While in some cases viruses directly infect endothelial cells, in other cases antibodies generated to the viruses target and activate the endothelial cells [132].

Viruses are known to also induce various inflammatory molecular pathways. For example, in one study which is incorporated by reference, human umbilical vein endothelial cells (HUVEC) were infected in vitro with clinical strains of HCMV and the resulting changes in adhesion molecule expression were quantified by histology and flow cytometric analysis. On HUVEC, surface expression of vascular cell adhesion molecule-1 and E-selectin was induced de novo on HCMV infection and intercellular adhesion molecule-1 expression was increased by >200%. On hvSMC, intercellular adhesion molecule-1 surface expression induced de novo, although vascular cell adhesion molecule-1 and E-selectin were not changed. Expression of major histocompatibility complex (MHC) class II, lymphocyte-function associated antigen 3 (LFA-3; CD58), and CD40 was not altered by HCMV infection in either cell type. In partially infected cultures, up-regulation of surface molecules also occurred on noninfected cells, suggesting a paracrine mechanism via a soluble factor. Expression of surface molecules could be enhanced in noninfected HUVEC and hvSMC by incubation with virus-free conditioned supernatant from HCMV-infected cells or by coincubation in transwells with infected cells. The responsible agent could be identified as IL-interleukin-(IL) 1beta by detection of de novo secretion of IL-1beta by HCMV-infected cells and by prevention of adhesion molecule up-regulation after addition of an IL-1-converting enzyme inhibitor or IL-1 receptor antagonist [133].

It is known that some of the endothelial abnormalities induced by viral infections are reminiscent of those induced by cytokine or immunotherapy administration [134].

For the purpose of understanding the disclosure, unless defined otherwise, all technical and scientific terms used herein have the same meaning as commonly understood by one of ordinary skill in the art to which this invention belongs.

It is to be understood by the description provided herein that this invention is not limited to the particular methodology, protocols, cell lines or type of stem cell, constructs, additives, and reagents described herein. It is also to be understood that the terminology used herein is for the purpose of describing particular embodiments only, and is not intended to limit the scope of the present invention which will be limited only by the appended claims.

The term “stem cell” refers to any multipotent or pluripotent cell, traditional stem cells, progenitor cells, preprogenitor cells, and reserve cells. These cells include Mesenchymal Stem Cells, Hematopoietic Stem Cells, Endothelial Stem Cells, and Pericytes. The term is used interchangeably with and may mean progenitor cell. A stem cell may be derived from an adult organism or from a cell line, or from an embryonic organism. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, and the like. Descriptions of stem cells, including method for isolating and culturing them, may be found in, among other places, Embryonic Stem Cells, Methods and Protocols, Turksen ed., Humana Press, 2002.

The term “adult” as used herein refers to any non-embryonic organism. For example the term “adult adipose-derived regenerative cell,” refers to an adipose-derived regenerative cell, other than that obtained from an embryo.

The term “embryo” as used herein refers to any multicellular diploid eukaryote during development, until birth or hatching. The term “embryonic stem cell” refers to a pluripotent cell derived from the inner cell mass of a blastocyst.

The term “mesenchymal stem cell” refers to any multipotent stromal cell derived from, for example and without limitation, umbilical cord blood, adipose tissue, muscle, corneal stroma, and dental pulp that can differentiate into cells such as, including but not limited to, osteoblasts, chondrocytes, and adipocytes.

The term “adipose-derived regenerative cell” (ADRC) is used interchangeably with adipose stem cells (ASC) herein and refers to adult cells that originate from adipose tissue. ADRC are a heterologous population of cells comprising at least one of the following population of cells; adult stem cells, vascular endothelial cells, vascular smooth muscle cells, endothelial cells, mesenchymal stem cells, stems, pericytes and additional other cell types.

In some embodiments, ADRC refers to a substantially pure population of adipose-derived stem cells. ADRC can be easily harvested from adipose tissue and are substantially free of adipocytes and red blood cells and clonal populations of connective tissue stem cells. The stromal vascular fraction cells are substantially devoid of extracellular matrix material from adipose tissue. ADRC may also be referred to as adipose-derived stem/stromal cells (ASCs), adipose-derived adult stem (ADAS) cells, adipose-derived adult stromal cells, adipose-derived stromal cells, adipose stromal cells, adipose mesenchymal cells, adipose-derived mesenchymal stem cells, lipoblasts, pericytes, preadipocytes, and processed lipoaspirate cells.

The term “adipose” as used herein refers to any fat tissue from a subject. The terms “adipose” and “adipose tissue” are used interchangeably herein. The adipose tissue may be brown fat, white fat or yellow fat or white adipose tissue, derived from subcutaneous, omental/visceral, mammary, gonadal, or other adipose tissue site. The adipose tissue has adipocytes and stroma. Adipose tissue is found throughout the body of an animal. For example, in mammals, adipose tissue is present in the omentum, bone marrow, subcutaneous space, and surrounding most organs. Such cells may comprise a primary cell culture or an immortalized cell line. The adipose tissue may be from any organism having fat tissue. Preferably, the adipose tissue is human; most preferably, the adipose tissue is derived from the individual in need of treatment for a penile defect. A convenient source of adipose tissue is from liposuction surgery, however, the source of adipose tissue or the method of isolation of adipose tissue is not critical to the invention, and acquisition of adipose tissue by any means may adequately provide tissue and stem cells for the present invention.

The term “tissue” as used herein is a broad term that is applied to any group of cells that perform specific functions, and includes in some instances whole organs and/or part of organs. A tissue need not form a layer, and thus encompasses a wide range of tissue, including adipose tissue derived from any source in an organism. Preferably, the tissue is derived from a mammal. Most preferably, the tissue is derived from the individual in need of treatment for a penile defect.

The term “implant” as used herein refers to any method for transferring a population of cells or cell mass into a subject, including by surgical implantation (incision into the tissue of interest and deposition therein) and injection by a syringe, needle, cannula, or the like of any suitable gauge. An implant as used herein can comprise genetically modified cells, as well as cells differentiated from other cells, such as stem cells, progenitors, and the like, as well as adipose cells or tissue.

The term “corpus cavemosum” of the penis refers to one of a pair of sponge-like regions of erectile tissue which contain most of the blood in the penis during penile erection. Generally, the two corpus cavemosum and a corpus spongiosum are three expandable erectile tissues along the length of the penis which fill with blood during erection. The term “corpus” is used interchangeably herein with corporal, corporeal and corporic, which are terms used to describe tissues which are derived from the corpora cavemosum or which can be developed, differentiated, or altered by natural or artificial means into corpora cavernosum tissue. The term “cavernosum” is used interchangeably herein as cavernae, corporum, cavernosum, or cavemosorum penis, and refers to the caverns of corpora cavernosa (or one of the two corpus cavernosum) of the penis or the dilatable spaces within the corpus cavemosum of the penis, which fill with blood and become distended with erection.

The term “tunica albuginea” refers to the fibrous tissue covering, or enveloping, the corpora cavemosa of the penis. This tissue consists of elastin and collagen. The term “Buck's fascia” refers to the layer of fascia covering the penis, including the tunica albuginea.

The terms “subject” and “individual” are used interchangeably herein, and refer to an animal, for example a human, from whom stem cells, for example penile stem cells can be harvested, or a subject into whom tissue can be transplanted for treatment, for example treatment for penile defects, using the compositions and methods described herein. For treatment of conditions or disease states which are specific for a specific animal such as a human subject, the term “subject” refers to that specific animal. In some embodiments, the subject is a human subject. It is possible in embodiments of this invention that recipient subjects are of a different mammalian subject than the donor subject.

In one embodiment of the invention stem cells are administered together with antioxidants. Use of antioxidants may be utilized in accordance with other work that has used antioxidants to inhibit the pathological effects of viral infection on endothelial cells. For example, in one study which is incorporated by reference, investigators supplemented the diet of 10 HIV-seropositive subjects with 100 microg selenium daily, 11 subjects with 30 mg beta-carotene twice daily while 15 subjects were not supplemented. Plasma was obtained at outset and after a year, and tested by ELISA for endothelial cell, platelet and inflammatory markers. The non-supplemented patients experienced increases in von Willebrand factor and soluble thrombomodulin (both p<0.01). There were no changes in any of the indices in the patients taking selenium or beta-carotene. Increased von Willebrand factor and soluble thrombomodulin in the non-supplemented patients imply increased damage to the endothelium over the year of the study. Therefore it was interpreted that the lack of increase in the patients taking antioxidants as evidence of the protection of the endothelium by these agents [135].

In one embodiment of the invention administration of regenerative cells is performed in a patient suffering from diabetes and/or a viral or bacterial infection. The association between inflammation, diabetes and sexual dysfunction was previously discussed and is incorporated by reference. In one study, 57 diabetic patients, and 33 without erectile dysfunction, were enrolled in a case-control study. Both groups of patients consists of type 1 and type 2 diabetics. Serum antibodies against cytomegalovirus and C. pneumoniae and markers of inflammation, including high-sensitivity C-reactive protein and fibrinogen, were measured. Adjusted odds ratios for erectile dysfunction in cytomegalovirus IgG, C. pneumoniae IgG and C. pneumoniae IgA seropositive men were 2.4 (95% CI; 1.0-6.0), 3.0 (95% CI; 1.2-8.1) and 1.8 (95% CI; 0.7-4.6), respectively. Odds ratios for the highest tertiles of high-sensitivity C-reactive protein and fibrinogen concentrations compared to the lowest tertile were 4.3 (95% CI; 1.4-13.1) and 6.6 (95% CI; 2.1-21.2), respectively. Elevated high-sensitivity C-reactive protein or fibrinogen serum levels and infection with cytomegalovirus or C. pneumoniae were associated with erectile dysfunction in diabetes. The relation between cytomegalovirus and erectile dysfunction is markedly present in patients with elevated high-sensitivity C-reactive protein and fibrinogen levels, suggesting a modifying effect by the inflammation [136].

In one embodiment of the invention agents possessing anti-inflammatory activity are administered prior to, and/or concurrent with, and/or subsequent to administration of regenerative cells in a patient suffering from sexual dysfunction associated with viral infection. Demonstration of vascular repair in the context of viral infection was previously shown with pentoxifylline and incorporated by reference. anti-inflammatory drug pentoxifylline to reduce systemic inflammation and improve endothelial function, measured by flow-mediated dilation of the brachial artery, in HIV-infected patients not requiring antiretroviral therapy. Pentoxifylline significantly reduced circulating levels of vascular cell adhesion molecule-1 and interferon-gamma-induced protein and significantly improved endothelial function during the 8-week trial. Pentoxifylline may reverse HIV-related endothelial dysfunction by directly inhibiting the endothelial leukocyte adhesion pathway [137].

In some embodiments of the invention increased endothelial progenitor cells in circulation are utilized as a means of overcoming viral induced endothelial dysfunction in association with administration of regenerative cells to repair tissue associated with sexual function. Reduction in endothelial progenitor cells has been reported as a potential cause of viral induced endothelial dysfunction [138]. Means of mobilizing endothelial progenitor cells are known in the art and incorporated by reference. Treatment of sexual dysfunction may also be performed by mobilization of endogenous stem cells. Stem cells” as used herein are cells that are not terminally differentiated and are therefore able to produce cells of other types. Characteristic of stem cells is the potential to develop into mature cells that have particular shapes and specialized functions, such as heart cells, skin cells, or nerve cells. Stem cells are divided into three types, including totipotent, pluripotent, and multipotent. “Totipotent stem cells” can grow and differentiate into any cell in the body and thus, can form the cells and tissues of an entire organism. “Pluripotent stem cells” are capable of self-renewal and differentiation into more than one cell or tissue type. “Multipotent stem cells” are clonal cells that are capable of self-renewal, as well as differentiation into adult cell or tissue types. Multipotent stem cell differentiation may involve an intermediate stage of differentiation into progenitor cells or blast cells of reduced differentiation potential, but are still capable of maturing into different cells of a specific lineage. The term “stem cells”, as used herein, refers to pluripotent stem cells and multipotent stem cells capable of self-renewal and differentiation. “Bone marrow-derived stem cells” are the most primitive stem cells found in the bone marrow which can reconstitute the hematopoietic system, possess endothelial, mesenchymal, and pluripotent capabilities. Stem cells may reside in the bone marrow, either as an adherent stromal cell type, or as a more differentiated cell that expresses CD34, either on the cell surface or in a manner where the cell is negative for cell surface CD34. “Adult stem cells” are a population of stem cells found in adult organisms with some potential for self-renewal and are capable of differentiation into multiple cell types. Other examples of stem cells are marrow stromal cells (MSCs), HSC, multipotent adult progenitor cells (MAPCs), very small embryonic-like stem cells (VSEL), epiblast-like stem cell (ELSC) or blastomere-like stem cell (BLSC).

In some embodiments stem cells may be mobilized using various natural ingredients. In various embodiments, the dosage of the each of the one or more mobilization agents in the composition can include 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. For example, a representative set of dosages in the composition are shown in Table 1. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Aloe or extracts thereof, Polygonum multiflorum or extracts thereof, Lycium barbarum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items. In certain embodiments, Aloe is Aloe macroclada. In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In various embodiments, the total dosage amount is administered daily for one or more days, or multiple times in a single day. The present invention further provides a method of enhancing the trafficking of stem cells in a subject. In one embodiment, the level of trafficking of stem cells relates to the number of circulating hematopoietic stem cells (HSCs) in the peripheral blood of a subject. In another embodiment, the level of trafficking of stem cells relates to the number of circulating bone marrow-derived stem cells in the peripheral blood of a subject. In various embodiments, enhancing the trafficking of stem cells in a subject, includes administering a therapeutically effective amount of a mobilization agent, thereby increasing the release, circulation, homing and/or migration of stem cells in the subject, regardless of the route of administration. In another embodiment, the method provided herein enhances the trafficking of stem cells in a subject, including administering a therapeutically effective amount of a composition containing one or more of the following components selected from the group including: Aloe or extracts thereof, Polygonum multiflorum or extracts thereof, Lycium barbarum or extracts thereof, colostrum or extracts thereof, spirulina or extracts thereof, Arthrospira platensis or extracts thereof, Arthrospira maxima or extracts thereof, fucoidan or extracts thereof, Chordaria cladosiphon or extracts thereof, Hericium erinaceus or extracts thereof, Ganoderma lucidum or extracts thereof, and/or Cordyceps sinensis or extracts thereof, thereby enhancing the trafficking of stem cells in the subject. In one embodiment, enhancement of stem cell trafficking may be measured by assaying the response of stem cells to a particular dose of a composition containing one or more of the following components selected from the group including: Aloe or extracts thereof, Lycium barbarum or extracts thereof, colostrum or extracts thereof, spirulina or extracts thereof, Arthrospira platensis or extracts thereof, Arthrospira maxima or extracts thereof, fucoidan or extracts thereof, Chordaria cladosiphon or extracts thereof, Hericium erinaceus or extracts thereof, Ganoderma lucidum or extracts thereof, and/or Cordyceps sinensis or extracts thereof, thereby enhancing the trafficking of stem cells in the subject. In another embodiment, a method of enhancing the trafficking of stem cells in a subject includes a transient increase in the population of circulating stem cells, such as stem cells following administration of a mobilization agent. In one embodiment, the stem cells are hematopoietic stem cells (HSGs). In another embodiment, the stem cells are bone marrow-derived stem cells. In various embodiments, the stem cells are CD45.sup.dim CD34.sup.+, CD34.sup.+, CD34.sup.+KDR.sup.−, or CD45−CD31+KDR+, CD34+CD133−, CD34+CD133+, or express various sub-combinations of these markers. In another embodiment, the administration of an extract of a mobilization agent leads to an increase in CXCR4 expression on circulating stem cells. In one embodiment, providing a mobilization agent to a subject will enhance release of that subject's stem cells within a certain time period, such as less than 12 days, less than 6 days, less than 3 days, less than 2, or less than 1 days. In an alternative embodiment, the time period is less than 12 hours, 6 hours, less than about 4 hours, less than about 2 hours, or less than about 1 hour following administration. In various embodiments, release of stem cells into the circulation from about 1, 2, or 3 hours following administration. In another embodiment, released stem cells enter the circulatory system and increase the number of circulating stem cells within the subject's body. In another embodiment, the percentage increase in the number of circulating stem cells compared to a normal baseline may about 25%, about 50%, about 100% or greater than about 100% increase as compared to a control. In one embodiment, the control is a base line value from the same subject. In another embodiment, the control is the number of circulating stem cells in an untreated subject, or in a subject treated with a placebo or a pharmacological carrier.

In another embodiment, treatment of sexual dysfunction is accomplished by administration of a regenerative cell alone, and or together with applying a a method of enhancing the trafficking of stem cells in a subject includes a transient decrease in the number of circulating stem cells within the subject's body. In another embodiment, a method of enhancing the trafficking of stem cells in a subject includes inducing a transient decrease in the population of circulating stem cells, such as stem cells. In one embodiment, the stem cells are hematopoietic stem cells (HSGs). In another embodiment, the stem cells are bone marrow-derived stem cells. In various embodiments, the stem cells are CD45.sup.dim CD34.sup.+, CD34.sup.+, CD34.sup.+KDR.sup.−, or CD45−CD31+KDR+, CD34+CD133−, CD34+CD133+, or express various sub-combinations of these markers. In another embodiment, the administration of an extract of a mobilization agent leads to an increase in CXCR4 expression on circulating stem cells. In one embodiment, providing a mobilization agent to a subject will enhance migration of that subject's stem cells within a certain time period, such as less than about 5 hours, less than about 4 hours, less than about 2 hours, or less than about 1 hour following administration. In other embodiments, the mobilization agent is colostrum, mushroom polysaccharides including Cordyceps sinensis, Hericium erinaceus, Ganoderma lucidum, fucoidan including Chordaria cladosiphon, spirulina, including Arthrospira platensis, and/or Arthrospira maxima. In various embodiments, the percentage decrease in the number of circulating stem cells compared to a normal baseline may about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier. In one embodiment, administration of a mobilization agent results in the migration of stem cells from the circulation to tissues from about 1 to about 3 hours following administration. Circulating stem cells will leave the circulatory system, thus decreasing the number of circulating stem cells within the subject's body. The percentage decrease in the number of circulating stem cells compared to a normal baseline may be about 15%, about 30%, about 50% or greater than about 75% decrease as compared to a control. In one embodiment, the control is a base line value from the same subject. In another embodiment, the control is the number of circulating stem cells in an untreated subject, or in a subject treated with a placebo or a pharmacological carrier. In another embodiment, administration a mobilization agent increases the rate of homing of stem cells measured by a transient decrease in the number of circulating stem cells within the subject's body. The percentage decrease in the number of circulating stem cells compared to a normal baseline may be about 25%, about 50%, about 75%, or even about 100% as compared to a control. In one embodiment, the control is a base line value from the same subject. In another embodiment, the control is the number of circulating stem cells in an untreated subject, or in a subject treated with a placebo or a pharmacological carrier. In another embodiment, the administration of an extract of a mobilization agent leads to an increase in CXCR4 expression on circulating stem cells. In various embodiments, administering a therapeutically effective amount of a composition includes oral administration of a dosage containing one or more mobilization agents in the amount of 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of the mobilization agents. For example, the one or more mobilization agents in the compositions can be combined at each of these variable dosage amounts. In various embodiments, the composition includes 1-5, 5-10, 10-25, 25-50, 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000 mg or more of Aloe or extracts thereof, Polygonum multiflorum or extracts thereof, Lycium barbarum, colostrum, mushroom polysaccharides (e.g., Cordyceps sinensis, Hericium erinaceus (Lion's mane), Ganoderma lucidum (Reishi)), fucoidan (optionally extracted from algaes, e.g., Undaria pinnatifida, Chordaria cladosiphon (Limu)), spirulina (e.g., Arthrospira platensis, Arthrospira maxima), analogs thereof, derivatives thereof, extracts thereof, synthetic or pharmaceutical equivalents thereof, fractions thereof, and combinations of any of the foregoing items. In certain embodiments, Aloe is Aloe macroclada. In various embodiments, the dosages can contain one or more mobilization agents for a total amount of 50-250, 250-500, 500-750, 750-1000, 1000-2000, 2000-3000, 3000 mg or more. For example, in various embodiments, the pharmaceutical composition includes 750 mg or less of Aloe macroclada and 1000 mg or less of one or more of: Polygonum multiflorum or extracts thereof, Lycium barbarum or extracts thereof, colostrum or extracts thereof, spirulina or extracts thereof, fucoidan, Hericium erinaceus or extracts thereof, Ganoderma lucidum or extracts thereof, and/or Cordyceps sinensis or extracts thereof. In various embodiments, the total dosage amount is administered daily for one or more days, or multiple times in a single day.

In other embodiments endogenous EPC are mobilized by a procedure in which G-CSF is administered, any formulation of G-CSF or other stem cell proliferation agents may be included in the composition and administered into the patient. Examples of other stem cell proliferation agents may include, for example, AMD 3100, CXCR4 antagonist [139, 140], up regulator of metalloproteinase (MMP-9) expression, up regulator of VEGF, SDF-1, angiopoietin-1 over expression, granulocyte monocyte colony stimulating factor (GM-CSF), erythropoietin, 3-hydroxy-3-methylglutaryl coenzyme A reductase inhibitors, statins, peroxisome proliferator-activated receptor gamma agonists, placental growth factor, estrogen, VEGF-A, and/or VEGFR2. In an exemplary embodiment where G-CSF is administered, commercially available recombinant human G-CSF, for example, Neupogen1M may be used, NeulastalM, recombinant G-CSF, or G-CSF produced from hamster ovary cells. A single source of G-CSF, or a combination of derivatives and sources of G-CSF, may be used in the composition. In an embodiment, the G-CSF administered is a glycoprotein with a molecular weight of 19.6 KDa. The G-CSF may be introduced into to the patient in any suitable form or formulation. For example, the G-CSF may be formulated in pharmaceutically acceptable carriers or diluents such as physiological saline or a buffered salt solution.

In some situations clinical protocols are utilized from existing papers to provide concentrations and guidance for one practicing the current invention. For example, a patient suffering from erectile dysfunction after a COVID-19 infection may be treated by administration of stem cells or regenerative cells alone. On the other hand, the patient, under the current invention may be subjected to protocols to mobilize endogenous stem cells. Additionally, in some situations combinations of regenerative cells together with mobilization of autologous cells may be applied. Some examples of mobilization therapy are included to provide doses and protocols for therapeutic interventions in other conditions that may be applied to sexual dysfunction. Shephard et al performed a clinical trial utilizing healthy donors which were mobilized sequentially with the CXCR4 antagonist, AMD3100, and G-CSF. The number of EPCs and circulating angiogenic cells (CACs) in the blood and pheresis product was determined and the angiogenic capacity of each cell population assessed. Compared with baseline, treatment with AMD3100 or G-CSF increased the number of blood CACs 10.0-fold+/−4.4-fold and 8.8-fold+/−3.7-fold, respectively. The number of EPCs in the blood increased 10.2-fold+/−3.3-fold and 21.8-fold+/−5.4-fold, respectively. On a per cell basis, CACs harvested from G-CSF-mobilized blood displayed increased in vivo angiogenic potential compared with AMD3100-mobilized CACs. Mobilized EPCs displayed a greater proliferative capacity than EPCs isolated from baseline blood. Both CACs and EPCs were efficiently harvested by leukapheresis. Cryopreserved CACs but not EPCs retained functional activity after thawing. These data show that AMD3100 is a potent and rapid mobilizer of angiogenic cells and demonstrate the feasibility of obtaining and storing large numbers of angiogenic cells by leukapheresis [141]. In another study, Aortic grafts were performed in Brown Norway (BN, donor) and Lewis (Lew, recipient) rats. The recipient rats were treated with low molecular weight fucoidon (LMWF) (5 mg/kg/day) and sacrificed at 30 days. To determine the role of SDF-1 in mediating the effects of LMWF, a specific inhibitor of the SDF-1 receptor CXCR4, AMD 3100 (20 microg/kg/day), was used. The grafted segments were evaluated by morphometric (histochemical) analyses. Untreated aortic allografts exhibited severe intimal proliferation, indicative of TA. In contrast, LMWF treatment significantly prevented allograft intimal proliferation as compared with controls (5.7+/−3 vs. 66.2+/−6 microm, P<0.01) and permitted a normalization of the intima/media ratio (0.1+/−0.1 vs. 1.7+/−0.3, P<0.01). Further, LMWF treatment stimulated allograft reendothelialization, as evidenced by strong intimal endothelial nitric oxide synthase antibody and CD31 signals. Unexpectedly, AMD treatment failed to prevent the protective effect of LMWF on intimal thickening and AMD treatment alone was found to reduced intimal proliferation in allografts. We found that LMWF treatment reduced intimal thickness and induced the presence of an endothelial cell lining in the vascular graft at 30 days. Our findings may suggest a novel therapeutic strategy in the prevention of TA [142]. In another study, the effect of the effect of M-CSF treatment on infarct size and left ventricular (LV) remodeling after MI. MI was induced in C57BL/6J mice by ligation of the left coronary artery. Either recombinant human M-CSF or saline was administered for 5 consecutive days after MI induction. M-CSF treatment significantly reduced the infarct size (P<0.05) and scar formation (P<0.05) and improved the LV dysfunction (percent fractional shortening, P<0.001) after the MI. Immunohistochemistry revealed that M-CSF increased macrophage infiltration (F4/80) and neovascularization (CD31) of the infarct myocardium but did not increase myostem accumulation (alpha-smooth muscle actin). M-CSF mobilized CXCR4(+) cells into peripheral circulation, and the mobilized CXCR4(+) cells were then recruited into the infarct area in which SDF-1 showed marked expression. The CXCR4 antagonist AMD3100 deteriorated the infarction and LV function after the MI in the M-CSF-treated mice. In conclusion, M-CSF reduced infarct area and improved LV remodeling after MI through the recruitment of CXCR4(+) cells into the infarct myocardium by the SDF-1-CXCR4 axis activation; this suggests that the SDF-1-CXCR4 axis is as a potential target for the treatment of MI [143]. Mobilization of stem cells of the recipient was found therapeutic in a series of experiments by Roux et al in which Aortic transplants were made from balb/c donor to C57Bl/6 recipient mice. Three different mobilizing pharmacologic agents were used: low molecular weight fucoidan (LMWF), simvastatin, and AMD3100. The circulating levels of progenitor cells were found to be increased by all three treatments, as determined by flow cytometry. For each treatment, the design was: treated allografts, nontreated allografts, treated isografts, and nontreated isografts. After 21 d, morphometric and immunohistochemical analyses were performed. We found that the three treatments significantly reduced intimal proliferation, compared with nontreated allografts. This was associated with intimal re-endothelialization of the grafts. Further, in chimeric mice that had previously received GFP-transgenic bone marrow transplantation, GFP-positive cells were found in the vascular allograft intima, indicating that re-endothelialization was, at least partly, due to the recruitment of bone marrow-derived, presumably endothelial progenitor circulating cells. The authors concluded that In this aortic allograft model, three different mobilizing treatments were found to partially prevent vascular transplant rejection. Bone marrow-derived progenitor cells mobilized by the three treatments may play a direct role in the endothelial repair process and in the suppression of intimal proliferation [144]. Yao et al used atherosclerosis-prone mouse model in which hypercholesterolemia, one of the main factors affecting EPC homeostasis, is reversible (Reversa mice). In these mice, normalization of plasma lipids decreased atherosclerotic burden; however, plaque regression was incomplete. To explore whether endothelial progenitors contribute to atherosclerosis regression, bone marrow EPCs from a transgenic strain expressing green fluorescent protein (GFP) under the control of endothelial cell-specific Tie2 promoter (Tie2-GFP(+)) were isolated. These cells were then adoptively transferred into atheroregressing Reversa recipients where they augmented plaque regression induced by reversal of hypercholesterolemia. Advanced plaque regression correlated with engraftment of Tie2-GFP(+) EPCs into endothelium and resulted in an increase in atheroprotective nitric oxide and improved vascular relaxation. Similarly augmented plaque regression was also detected in regressing Reversa mice treated with the stem cell mobilizer AMD3100 which also mobilizes EPCs to peripheral blood. They concluded that correction of hypercholesterolemia in Reversa mice leads to partial plaque regression that can be augmented by AMD3100 treatment or by adoptive transfer of EPCs. This suggests that direct cell therapy or indirect progenitor cell mobilization therapy may be used in combination with statins to treat atherosclerosis [145]. Other examples of therapeutic mobilization for treatment of endothelial related issues such as atherosclerosis [146-157], heart failure [158-162], endothelial function [163-168], and angiogenesis [169-175], are incorporated by reference.

In one embodiment of the invention, regenerative cells are bone marrow cells. In other embodiments, the invention describes the useful of bone marrow mesenchymal cells. In yet other embodiments, the invention teaches the use of bone marrow mononuclear cells.

The underlying theme of the invention teaches the use of stem cells for the treatment of erectile dysfunction. Specific properties of stem cells, including bone marrow mononuclear cells that are suitable for use in practicing the current invention are: a) ability to both increase endothelial function, as well as induce neoangiogenesis; b) ability to prevent atrophy, as well as to differentiate into functional penile tissue; and c) ability to induce local resident stem/progenitor cells to proliferate through secretion of soluble factors, as well as via membrane bound activities. In one embodiment of the invention, stem cells cells are collected from an autologous patient, expanded ex vivo, and reintroduced into said patient at a concentration and frequency sufficient to cause therapeutic benefit in ED. Said stem cells are selected for ability to cause: neoangiogenesis, prevention of tissue atrophy, and regeneration of functional tissue.

When selecting stem cells for use in the practice of the current invention, several factors must be taken into consideration, such as: ability for ex vivo expansion without loss of therapeutic activity, ease of extraction, general potency of activity, and potential for adverse effects. Ex vivo expansion ability of stem cells can be measured using typical proliferation and colony assays known to one skilled in the art, while identification of therapeutic activity depends on functional assays that test biological activities such as: ability to support endothelial function, ability to protect neurons from degeneration/atrophy, and, ability to inhibit smooth muscle atrophy/degeneration. Assessment of therapeutic activity can also be performed using surrogate assays which detect markers associated with a specific therapeutic activity. Assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating therapeutic activity in vivo [176]. Additionally, secretion of factors that inhibit smooth muscle atrophy or neuronal dysfunction may also be used as a marker for identification of cells that are useful for practicing the current invention.

In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In some embodiments of the invention, cells are directly injected into the area of need, such as in the corpora cavernosa, in which case it will be desirable for said stem cells to be substantially differentiated, whereas in other embodiments, cells will be administered systemically and it this case with may be desirable for the administered cells to be less differentiated, so has to still possess homing activity to the area of need. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood stem are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

### REFERENCES

1. Sansone, A., et al., Addressing male sexual and reproductive health in the wake of COVID-19 outbreak. J Endocrinol Invest, 2021. 44(2): p. 223-231.
2. Bertolo, R., C. Cipriani, and P. Bove, Anosmia and ageusia: a piece of the puzzle in the etiology of COVID-19-related transitory erectile dysfunction. J Endocrinol Invest, 2021. 44(5): p. 1123-1124.
3. Sansone, A., et al., “Mask up to keep it up”: Preliminary evidence of the association between erectile dysfunction and COVID-19. Andrology, 2021. 9(4): p. 1053-1059.
4. Salama, N. and S. Blgozah, COVID-19 and Male Sexual Functioning: A report of 3 Recovered Cases and Literature Review. Clin Med Insights Case Rep, 2021. 14: p. 11795476211020593.
5. Nassau, D. E., et al., Impact of the SARS-CoV-2 virus on male reproductive health. BJU Int, 2021.
6. Kresch, E., et al., COVID-19 Endothelial Dysfunction Can Cause Erectile Dysfunction: Histopathological, Immunohistochemical, and Ultrastructural Study of the Human Penis. World J Mens Health, 2021. 39(3): p. 466-469.
7. Sansone, A. and E. A. Jannini, COVID-19 and Erectile Dysfunction: Endothelial Dysfunction and Beyond. World J Mens Health, 2021. 39(4): p. 820-821.
8. Malik, J., et al., Love in the time of COVID-19: a scoping review on male sexual health. J Community Hosp Intern Med Perspect, 2021. 11(4): p. 496-500.
9. Pal, M., et al., Severe Acute Respiratory Syndrome Coronavirus-2 (SARS-CoV-2): An Update. Cureus, 2020. 12(3): p. e7423.
10. Malik, Y. A., Properties of Coronavirus and SARS-CoV-2. Malays J Pathol, 2020. 42(1): p. 3-11.
11. Huang, P., et al., Nucleic acid visualization assay for Middle East Respiratory Syndrome Coronavirus (MERS-CoV) by targeting the UpE and N gene. PLoS Negl Trop Dis, 2021. 15(3): p. e0009227.
12. Rabets, A., et al., The Potential of Developing Pan-Coronaviral Antibodies to Spike Peptides in Convalescent COVID-19 Patients. Arch Immunol Ther Exp (Warsz), 2021. 69(1): p. 5.
13. Alshehri, M. A., et al., On the Prevalence and Potential Functionality of an Intrinsic Disorder in the MERS-CoV Proteome. Viruses, 2021. 13(2).
14. Park, B. K., et al., MERS-CoV and SARS-CoV-2 replication can be inhibited by targeting the interaction between the viral spike protein and the nucleocapsid protein. Theranostics, 2021. 11(8): p. 3853-3867.
15. Chen, J., et al., Development of A MERS-CoV Replicon Cell Line for Antiviral Screening. Virol Sin, 2021. 36(4): p. 730-735.
16. Abdelghany, T. M., et al., SARS-CoV-2, the other face to SARS-CoV and MERS-CoV: Future predictions. Biomed J, 2021. 44(1): p. 86-93.
17. Ansariniya, H., et al., Comparison of Immune Response between SARS, MERS, and COVID-19 Infection, Perspective on Vaccine Design and Development. Biomed Res Int, 2021. 2021: p. 8870425.
18. Saha, J., et al., A comparative genomics-based study of positive strand RNA viruses emphasizing on SARS-CoV-2 utilizing dinucleotide signature, codon usage and codon context analyses. Gene Rep, 2021. 23: p. 101055.
19. Lu, R., et al., Genomic characterisation and epidemiology of 2019 novel coronavirus: implications for virus origins and receptor binding. Lancet, 2020. 395(10224): p. 565-574.
20. Zhang, C., et al., A Novel Scoring System for Prediction of Disease Severity in COVID-19. Front Cell Infect Microbiol, 2020. 10: p. 318.
21. Liu, B., et al., Can we use interleukin-6 (IL-6) blockade for coronavirus disease 2019 (COVID-19)-induced cytokine release syndrome (CRS)? J Autoimmun, 2020. 111: p. 102452.
22. Salama, C., et al., Tocilizumab in Patients Hospitalized with Covid-19 Pneumonia. N Engl J Med, 2021. 384(1): p. 20-30.
23. Zhang, C., et al., Cytokine release syndrome in severe COVID-19: interleukin-6 receptor antagonist tocilizumab may be the key to reduce mortality. Int J Antimicrob Agents, 2020. 55(5): p. 105954.
24. Sivritepe, R., et al., The effect of interleukin-6 level at the time of hospitalisation on erectile functions in hospitalised patients with COVID-19. Andrologia, 2022. 54(1): p. e14285.
25. Bin Lim, K. and G. B. Brock, The Erectile Function Visual Analog Scale (EF-VAS): a disease-specific utility instrument for the assessment of erectile function. Can J Urol, 2006. 13(2): p. 3026.
26. Karakiewicz, P., et al., Reliability of remembered International Index of Erectile Function domain scores in men with localized prostate cancer. Urology, 2005. 65(1): p. 131-5.
27. Kleinman, K. P., et al., A new surrogate variable for erectile dysfunction status in the Massachusetts male aging study. J Clin Epidemiol, 2000. 53(1): p. 71-8.
28. Foresta, C., et al., Erectile dysfunction: symptom or disease? J Endocrinol Invest, 2004. 27(1): p. 80-95.
29. Aversa, A., et al., Phosphodiesterase 5 inhibitors in the treatment of erectile dysfunction. Curr Pharm Des, 2006. 12(27): p. 3467-84.
30. http://www.faqs.org/patents/app/20090311223.
31. Wespes, E., et al., EAU Guidelines on erectile dysfunction: an update. Eur Urol, 2006. 49(5): p. 806-15.
32. Cour, F., et al., [Recommendations to general practice doctors for first line management of erectile dysfunction]. Prog Urol, 2005. 15(6): p. 1011-20.
33. Montague, D. K., et al., Chapter 1: The management of erectile dysfunction: an AUA update. J Urol, 2005. 174(1): p. 230-9.
34. Fraunfelder, F. W., H. D. Pomeranz, and R. A. Egan, Nonarteritic anterior ischemic optic neuropathy and sildenafil. Arch Ophthalmol, 2006. 124(5): p. 733-4.
35. Evans, R. W., Sildenafil can trigger cluster headaches. Headache, 2006. 46(1): p. 173-4.
36. Schwarz, E. R. and J. Rodriguez, Sex and the heart. Int J Impot Res, 2005. 17 Suppl 1: p. S4-6.
37. Rashid, A., The efficacy and safety of PDE5 inhibitors. Clin Cornerstone, 2005. 7(1): p. 47-56.
38. Shinlapawittayatorn, K., S. Chattipakorn, and N. Chattipakorn, Effect of sildenafil citrate on the cardiovascular system. Braz J Med Biol Res, 2005. 38(9): p. 1303-11.
39. Galie, N., et al., Sildenafil citrate therapy for pulmonary arterial hypertension. N Engl J Med, 2005. 353(20): p. 2148-57.
40. Kulkarni, S. K. and C. S. Patil, Phosphodiesterase 5 enzyme and its inhibitors: update on pharmacological and therapeutical aspects. Methods Find Exp Clin Pharmacol, 2004. 26(10): p. 789-99.
41. Czp, A., Citrulline, Viagra and BiDil—bad medicine. Altern Med Rev, 2005. 10(4): p. 265-7.
42. Min, J. K., et al., Prediction of coronary heart disease by erectile dysfunction in men referred for nuclear stress testing. Arch Intern Med, 2006. 166(2): p. 201-6.
43. Montorsi, P., et al., The artery size hypothesis: a macrovascular link between erectile dysfunction and coronary artery disease. Am J Cardiol, 2005. 96(12B): p. 19M-23M.
44. Borgquist, R., et al., Erectile dysfunction in healthy subjects predicts reduced coronary flow velocity reserve. Int J Cardiol, 2005.
45. Andersson, K. E. and G. Wagner, Physiology of penile erection. Physiological reviews, 1995. 75(1): p. 191-236.
46. Fournier, G. R., Jr., et al., Mechanisms of venous occlusion during canine penile erection: an anatomic demonstration. The Journal of urology, 1987. 137(1): p. 163-7.
47. Toda, N., K. Ayajiki, and T. Okamura, Nitric oxide and penile erectile function. Pharmacology & therapeutics, 2005. 106(2): p. 233-66.
48. Ravipati, G., et al., Type 5 phosphodiesterase inhibitors in the treatment of erectile dysfunction and cardiovascular disease. Cardiology in review, 2007. 15(2): p. 76-86.
49. Dussault, S., et al., Sildenafil increases endothelial progenitor cell function and improves ischemia-induced neovascularization in hypercholesterolemic apolipoprotein E-deficient mice. Hypertension, 2009. 54(5): p. 1043-9.
50. Foresta, C., et al., The PDE5 inhibitor sildenafil increases circulating endothelial progenitor cells and CXCR4 expression. The journal of sexual medicine, 2009. 6(2): p. 369-72.
51. Foresta, C., et al., PDE-5 inhibitor, Vardenafil, increases circulating progenitor cells in humans. International journal of impotence research, 2005. 17(4): p. 377-80.
52. Foresta, C., et al., Relationship between vascular damage degrees and endothelial progenitor cells in patients with erectile dysfunction: effect of vardenafil administration and PDE5 expression in the bone marrow. European urology, 2007. 51(5): p. 1411-7; discussion 1417-9.
53. Foresta, C., et al., Effect of vardenafil on endothelial progenitor cells in hypogonadotrophic hypogonadal patients: role of testosterone treatment. Clinical endocrinology, 2009. 71(3): p. 412-6.
54. Ferrini, M. G., et al., Vardenafil prevents fibrosis and loss of corporal smooth muscle that occurs after bilateral cavernosal nerve resection in the rat. Urology, 2006. 68(2): p. 429-35.
55. Kovanecz, I., et al., Long-term continuous sildenafil treatment ameliorates corporal veno-occlusive dysfunction (CVOD) induced by cavernosal nerve resection in rats. International journal of impotence research, 2008. 20(2): p. 202-12.
56. Ozden, E., et al., Effect of sildenafil citrate on penile weight and physiology of cavernous smooth muscle in a post-radical prostatectomy model of erectile dysfunction in rats. Urology, 2011. 77(3): p. 761 el-7.
57. Padma-Nathan, H., A. McCullough, and C. Forest, Erectile dysfunction secondary to nerve-sparing radical retropubic prostatectomy: comparative phosphodiesterase-5 inhibitor efficacy for therapy and novel prevention strategies. Current urology reports, 2004. 5(6): p. 467-71.
58. Sanna, F., et al., Phosphodiesterase type 5 inhibitors facilitate noncontact erections in male rats: site of action in the brain and mechanism of action. The journal of sexual medicine, 2009. 6(10): p. 2680-9.
59. Edwards, J. F., W. J. Dodds, and D. O. Slauson, Coagulation changes in African swine fever virus infection. Am J Vet Res, 1984. 45(11): p. 2414-20.
60. Anderson, E. C., et al., Arachidonic acid metabolites in the pathophysiology of thrombocytopenia and haemorrhage in acute African swine fever. Res Vet Sci, 1987. 42(3): p. 387-94.
61. Villeda, C. J., et al., Haemostatic abnormalities in African swine fever a comparison of two virus strains of different virulence (Dominican Republic '78 and Malta '78). Arch Virol, 1993. 130(1-2): p. 71-83.
62. Villeda, C. J., et al., The role of fibrinolysis in the pathogenesis of the haemorrhagic syndrome produced by virulent isolates of African swine fever virus. Thromb Haemost, 1995. 73(1): p. 112-7.
63. Van Dam-Mieras, M. C., et al., Induction of endothelial cell procoagulant activity by cytomegalovirus infection. Thromb Res, 1987. 47(1): p. 69-75.
64. van Dam-Mieras, M. C., et al., The procoagulant response of cytomegalovirus infected endothelial cells. Thromb Haemost, 1992. 68(3): p. 364-70.
65. Sabate, M., et al., Acute rejection, cytomegalovirus infection and endothelial dysfunction early after heart transplantation. Transplant Proc, 1995. 27(4): p. 2346-8.
66. Bertrand, M. E. and C. Bauters, Cytomegalovirus infection and coronary restenosis. Circulation, 1999. 99(10): p. 1278-9.
67. Rao, N. A., J. Zhang, and S. Ishimoto, Role of retinal vascular endothelial cells in development of CMV retinitis. Trans Am Ophthalmol Soc, 1998. 96: p. 111-23; discussion 124-6.
68. Kas-Deelen, A. M., et al., Pulmonary diffusion abnormalities in relation to cytomegalovirus antigenemia and cytomegalic endothelial cells in blood. Transpl Int, 2001. 14(3): p. 180-3.
69. Rott, D., et al., Serum of cytomegalovirus-infected mice induces monocyte chemoattractant protein-1 expression by endothelial cells. J Infect Dis, 2001. 184(9): p. 1109-13.
70. Reinhardt, B., et al., Human cytomegalovirus infection in human renal arteries in vitro. J Virol Methods, 2003. 109(1): p. 1-9.
71. Weis, M., et al., Cytomegalovirus infection impairs the nitric oxide synthase pathway: role of asymmetric dimethylarginine in transplant arteriosclerosis. Circulation, 2004. 109(4): p. 500-5.
72. Yamamoto-Tabata, T., et al., Human cytomegalovirus interleukin-10 downregulates metalloproteinase activity and impairs endothelial cell migration and placental cytotrophoblast invasiveness in vitro. J Virol, 2004. 78(6): p. 2831-40.
73. Shen, Y. H., et al., Human cytomegalovirus causes endothelial injury through the ataxia telangiectasia mutant and p53 DNA damage signaling pathways. Circ Res, 2004. 94(10): p. 1310-7.
74. Petrakopoulou, P., et al., Cytomegalovirus infection in heart transplant recipients is associated with impaired endothelial function. Circulation, 2004. 110(11 Suppl 1): p. 11207-12.
75. Rahbar, A. and C. Soderberg-Naucler, Human cytomegalovirus infection of endothelial cells triggers platelet adhesion and aggregation. J Virol, 2005. 79(4): p. 2211-20.
76. Bentz, G. L. and A. D. Yurochko, Human CMV infection of endothelial cells induces an angiogenic response through viral binding to EGF receptor and beta1 and beta3 integrins. Proc Natl Acad Sci USA, 2008. 105(14): p. 5531-6.
77. Khoretonenko, M. V., et al., Cytomegalovirus infection leads to microvascular dysfunction and exacerbates hypercholesterolemia-induced responses. Am J Pathol, 2010. 177(4): p. 2134-44.
78. McDonald, D. M., Respiratory tract infections increase susceptibility to neurogenic inflammation in the rat trachea. Am Rev Respir Dis, 1988. 137(6): p. 1432-40.
79. Vercellotti, G. M., Proinflammatory and procoagulant effects of herpes simplex infection on human endothelium. Blood Cells, 1990. 16(1): p. 209-15; discussion 215-6.
80. Visser, M. R. and G. M. Vercellotti, Herpes simplex virus and atherosclerosis. Eur Heart J, 1993. 14 Suppl K: p. 39-42.
81. Chirathaworn, C., A. Pongpanich, and Y. Poovorawan, Herpes simplex virus 1 induced LOX-1 expression in an endothelial cell line, ECV 304. Viral Immunol, 2004. 17(2): p. 308-14.
82. Resnick-Roguel, N., et al., Envelope glycoprotein of avian hemangioma retrovirus induces a thrombogenic surface on human and bovine endothelial cells. J Virol, 1990. 64(8): p. 4029-32.
83. Cosgriff, T. M., Mechanisms of disease in Hantavirus infection: pathophysiology of hemorrhagic fever with renal syndrome. Rev Infect Dis, 1991. 13(1): p. 97-107.
84. Pensiero, M. N., et al., Hantaan virus infection of human endothelial cells. J Virol, 1992. 66(10): p. 5929-36.
85. Kraus, A. A., et al., Differential antiviral response of endothelial cells after infection with pathogenic and nonpathogenic hantaviruses. J Virol, 2004. 78(12): p. 6143-50.
86. Hayasaka, D., et al., Increased permeability of human endothelial cell line EA.hy926 induced by hantavirus-specific cytotoxic T lymphocytes. Virus Res, 2007. 123(2): p. 120-7.
87. Mackow, E. R. and I. N. Gavrilovskaya, Hantavirus regulation of endothelial cell functions. Thromb Haemost, 2009. 102(6): p. 1030-41.
88. Marsac, D., et al., Infection of human monocyte-derived dendritic cells by ANDES Hantavirus enhances pro-inflammatory state, the secretion of active MMP-9 and indirectly enhances endothelial permeability. Virol J, 2011. 8: p. 223.
89. Lei, H. Y., et al., Immunopathogenesis of dengue virus infection. J Biomed Sci, 2001. 8(5): p. 377-88.
90. Lin, C. F., et al., Antibodies from dengue patient sera cross-react with endothelial cells and induce damage. J Med Virol, 2003. 69(1): p. 82-90.
91. Talavera, D., et al., IL8 release, tight junction and cytoskeleton dynamic reorganization conducive to permeability increase are induced by dengue virus infection of microvascular endothelial monolayers. J Gen Virol, 2004. 85(Pt 7): p. 1801-1813.
92. Lin, C. F., et al., Autoimmune pathogenesis in dengue virus infection. Viral Immunol, 2006. 19(2): p. 127-32.
93. Lin, Y. S., et al., Molecular mimicry between virus and host and its implications for dengue disease pathogenesis. Exp Biol Med (Maywood), 2011. 236(5): p. 515-23.
94. Gutsche, I., et al., Secreted dengue virus nonstructural protein NS1 is an atypical barrel-shaped high-density lipoprotein. Proc Natl Acad Sci USA, 2011. 108(19): p. 8003-8.
95. Lafeuillade, A., et al., Endothelial cell dysfunction in HIV infection. J Acquir Immune Defic Syndr (1988), 1992. 5(2): p. 127-31.
96. Goerdt, S. and C. Sorg, Endothelial heterogeneity and the acquired immunodeficiency syndrome: a paradigm for the pathogenesis of vascular disorders. Clin Investig, 1992. 70(2): p. 89-98.
97. Moses, A. V. and J. A. Nelson, HIV infection of human brain capillary endothelial cells—implications for AIDS dementia. Adv Neuroimmunol, 1994. 4(3): p. 239-47.
98. Moses, A. V., et al., Human immunodeficiency virus infection of bone marrow endothelium reduces induction of stromal hematopoietic growth factors. Blood, 1996. 87(3): p. 919-25.
99. Witz, M., J. Lehmann, and Z. Korzets, Acute brachial artery thrombosis as the initial manifestation of human immunodeficiency virus infection. Am J Hematol, 2000. 64(2): p. 137-9.
100. Ullrich, C. K., J. E. Groopman, and R. K. Ganju, HIV-1 gp120- and gp160-induced apoptosis in cultured endothelial cells is mediated by caspases. Blood, 2000. 96(4): p.
101. Aukrust, P., et al., Persistently elevated levels of von Willebrand factor antigen in HIV infection. Downregulation during highly active antiretroviral therapy. Thromb Haemost, 2000. 84(2): p. 183-7.
102. Passalaris, J. D., K. A. Sepkowitz, and M. J. Glesby, Coronary artery disease and human immunodeficiency virus infection. Clin Infect Dis, 2000. 31(3): p. 787-97.
103. Stein, J. H., et al., Use of human immunodeficiency virus-1 protease inhibitors is associated with atherogenic lipoprotein changes and endothelial dysfunction. Circulation, 2001. 104(3): p. 257-62.
104. Kanmogne, G. D., R. C. Kennedy, and P. Grammas, HIV-1 gp120 proteins and gp160 peptides are toxic to brain endothelial cells and neurons: possible pathway for HIV entry into the brain and HIV-associated dementia. J Neuropathol Exp Neurol, 2002. 61(11): p. 992-1000.
105. Toborek, M., et al., HIV-Tat protein induces oxidative and inflammatory pathways in brain endothelium. J Neurochem, 2003. 84(1): p. 169-79.
106. de Gaetano Donati, K., et al., Increased soluble markers of endothelial dysfunction in HIV-positive patients under highly active antiretroviral therapy. AIDS, 2003. 17(5): p. 765-8.
107. Paladugu, R., et al., Hiv Tat protein causes endothelial dysfunction in porcine coronary arteries. J Vasc Surg, 2003. 38(3): p. 549-55; discussion 555-6.
108. Andras, I. E., et al., HIV-1 Tat protein alters tight junction protein expression and distribution in cultured brain endothelial cells. J Neurosci Res, 2003. 74(2): p. 255-65.
109. Shankar, S. S. and M. P. Dube, Clinical aspects of endothelial dysfunction associated with human immunodeficiency virus infection and antiretroviral agents. Cardiovasc Toxicol, 2004. 4(3): p. 261-9.
110. Cotter, B. R., Endothelial dysfunction in HIV infection. Curr HIV/AIDS Rep, 2006. 3(3): p. 126-31.
111. Miller, T. L., et al., Biomarkers of vascular dysfunction in children infected with human immunodeficiency virus-1. J Acquir Immune Defic Syndr, 2010. 55(2): p. 182-8.
112. Kapetanovic, S., et al., Relationships between markers of vascular dysfunction and neurodevelopmental outcomes in perinatally HIV-infected youth. AIDS, 2010. 24(10): p. 1481-91.
113. Musselwhite, L. W., et al., Markers of endothelial dysfunction, coagulation and tissue fibrosis independently predict venous thromboembolism in HIV. AIDS, 2011. 25(6): p. 787-95.
114. Nzerue, M. C., Lassa fever: review of virology, immunopathogenesis, and algorithms for control and therapy. Cent Afr J Med, 1992. 38(6): p. 247-52.
115. Soilu-Hanninen, M., et al., Semliki Forest virus infects mouse brain endothelial cells and causes blood-brain barrier damage. J Virol, 1994. 68(10): p. 6291-8.
116. Bi, Z., et al., Vesicular stomatitis virus infection of the central nervous system activates both innate and acquired immunity. J Virol, 1995. 69(10): p. 6466-72.
117. Visseren, F. L., et al., Procoagulant activity of endothelial cells after infection with respiratory viruses. Thromb Haemost, 2000. 84(2): p. 319-24.
118. Marchesi, S., et al., Acute inflammatory state during influenza infection and endothelial function. Atherosclerosis, 2005. 178(2): p. 345-50.
119. Lippi, G., M. Franchini, and E. J. Favaloro, Influenza and cardiovascular disease: does swine-origin, 2009 H1N1 flu virus represent a risk factor, an acute trigger, or both? Semin Thromb Hemost, 2010. 36(1): p. 49-58.
120. Van Thiel, D. H., et al., Modulation of endothelial cell inflammatory integrins and stress markers with rh-factor VIIa in patients with advanced chronic hepatitis C. J Viral Hepat, 2003. 10(4): p. 310-7.
121. de Castro, I. F., et al., Hepatitis C virus infection is associated with endothelial dysfunction in HIV/hepatitis C virus coinfected patients. AIDS, 2010. 24(13): p. 2059-67.
122. Pawlak, K., M. Mysliwiec, and D. Pawlak, Hepatitis C virus seropositivity and TNF superfamily receptors: sCD40, sFas—the new putative determinants of endothelial dysfunction in haemodialysis patients. Thromb Res, 2010. 126(5): p. 393-8.
123. Gomez, R. M., et al., Endothelial cell function alteration after Junin virus infection. Thromb Haemost, 2003. 90(2): p. 326-33.
124. Pozner, R. G., et al., Junin virus infection of human hematopoietic progenitors impairs in vitro proplatelet formation and platelet release via a bystander effect involving type I IFN signaling. PLoS Pathog, 2010. 6(4): p. e1000847.
125. Selinka, H. C., et al., Virus-receptor interactions of coxsackie B viruses and their putative influence on cardiotropism. Med Microbiol Immunol, 2004. 193(2-3): p. 127-31.
126. Choy, J. C., et al., Coxsackievirus B3 infection compromises endothelial-dependent vasodilation of coronary resistance arteries. J Cardiovasc Pharmacol, 2004. 43(1): p. 39-47.
127. Dalpke, A. H., R. Thomssen, and K. Ritter, Oxidative injury to endothelial cells due to Epstein-Barr virus-induced autoantibodies against manganese superoxide dismutase. J Med Virol, 2003. 71(3): p. 408-16.
128. Liu, R., et al., KSHV-induced notch components render endothelial and mural cell characteristics and cell survival. Blood, 2010. 115(4): p. 887-95.
129. Hansen, A., et al., KSHV-encoded miRNAs target MAF to induce endothelial cell reprogramming. Genes Dev, 2010. 24(2): p. 195-205.
130. Schmidt-Lucke, C., et al., Interferon beta modulates endothelial damage in patients with cardiac persistence of human parvovirus b19 infection. J Infect Dis, 2010. 201(6): p. 936-45.
131. Drew, C. P., et al., Bluetongue virus infection alters the impedance of monolayers of bovine endothelial cells as a result of cell death. Vet Immunol Immunopathol, 2010. 136(1-2): p. 108-15.
132. Lin, C. F., et al., Expression of cytokine, chemokine, and adhesion molecules during endothelial cell activation induced by antibodies against dengue virus nonstructural protein 1. J Immunol, 2005. 174(1): p. 395-403.
133. Dengler, T. J., et al., Cytomegalovirus infection of vascular cells induces expression of pro-inflammatory adhesion molecules by paracrine action of secreted interleukin-1 beta. Transplantation, 2000. 69(6): p. 1160-8.
134. Wimer, B. M., Implications of the analogy between recombinant cytokine toxicities and manifestations of hantavirus infections. Cancer Biother Radiopharm, 1998. 13(3): p. 193-207.
135. Constans, J., et al., Effect of the antioxidants selenium and beta-carotene on HIV-related endothelium dysfunction. Thromb Haemost, 1998. 80(6): p. 1015-7.
136. Blans, M. C., et al., Infection induced inflammation is associated with erectile dysfunction in men with diabetes. Eur J Clin Invest, 2006. 36(7): p. 497-502.
137. Gupta, S. K., et al., Anti-inflammatory treatment with pentoxifylline improves HIV-related endothelial dysfunction: a pilot study. AIDS, 2010. 24(9): p. 1377-80.
138. Lopez, M., et al., Short communication high risk of endothelial dysfunction in HIV individuals may result from deregulation of circulating endothelial cells and endothelial progenitor cells. AIDS Res Hum Retroviruses, 2012. 28(7): p. 656-9.
139. De Clercq, E., The bicyclam AMD3100 story. Nat Rev Drug Discov, 2003. 2(7): p. 581-7.
140. Liles, W. C., et al., Mobilization of hematopoietic progenitor cells in healthy volunteers by AMD3100, a CXCR4 antagonist. Blood, 2003. 102(8): p. 2728-30.
141. Shepherd, R. M., et al., Angiogenic cells can be rapidly mobilized and efficiently harvested from the blood following treatment with AMD3100. Blood, 2006. 108(12): p. 3662-7.
142. Freguin-Bouilland, C., et al., Low molecular weight fucoidan prevents neointimal hyperplasia after aortic allografting. Transplantation, 2007. 83(9): p. 1234-41.
143. Morimoto, H., et al., Bone marrow-derived CXCR4+ cells mobilized by macrophage colony-stimulating factor participate in the reduction of infarct area and improvement of cardiac remodeling after myocardial infarction in mice. Am J Pathol, 2007. 171(3): p. 755-66.
144. Roux, N., et al., Progenitor cell mobilizing treatments prevent experimental transplant arteriosclerosis. J Surg Res, 2012. 176(2): p. 657-65.
145. Yao, L., et al., Bone marrow endothelial progenitors augment atherosclerotic plaque regression in a mouse model of plasma lipid lowering. Stem Cells, 2012. 30(12): p. 2720-31.
146. George, J., et al., Circulating endothelial progenitor cells in patients with unstable angina: association with systemic inflammation. Eur Heart J, 2004. 25(12): p. 1003-8.
147. Melo, L. G., et al., Endothelium-targeted gene and cell-based therapies for cardiovascular disease. Arterioscler Thromb Vasc Biol, 2004. 24(10): p. 1761-74.
148. Povsic, T. J. and E. D. Peterson, Progenitor cell therapy of ischemic heart disease: the new frontier. Future Cardiol, 2005. 1(1): p. 87-98.
149. Werner, N. and G. Nickenig, Clinical and therapeutical implications of EPC biology in atherosclerosis. J Cell Mol Med, 2006. 10(2): p. 318-32.
150. Arai, M., et al., Granulocyte colony-stimulating factor: a noninvasive regeneration therapy for treating atherosclerotic peripheral artery disease. Circ J, 2006. 70(9): p. 1093-8.
151. Stellos, K. and M. Gawaz, Platelets and stromal cell-derived factor-1 in progenitor cell recruitment. Semin Thromb Hemost, 2007. 33(2): p. 159-64.
152. Hristov, M., et al., Adult progenitor cells in vascular remodeling during atherosclerosis. Biol Chem, 2008. 389(7): p. 837-44.
153. Kocaman, S. A., et al., Endothelial progenitor cells (CD34+KDR+) and monocytes may provide the development of good coronary collaterals despite the vascular risk factors and extensive atherosclerosis. Anadolu Kardiyol Derg, 2011. 11(4): p. 290-9.
154. Mueller, C., et al., Darbepoetin improves endothelial function and increases circulating endothelial progenitor cell number in patients with coronary artery disease. Heart, 2011. 97(18): p. 1474-8.
155. Tousoulis, D., et al., Infusion of lin-/sca-1+ and endothelial progenitor cells improves proinflammatory and oxidative stress markers in atherosclerotic mice. Int J Cardiol, 2013. 167(5): p. 1900-5.
156. Du, F., et al., Endothelial progenitor cells in atherosclerosis. Front Biosci (Landmark Ed), 2012. 17: p. 2327-49.
157. Tousoulis, D., et al., Effects of direct infusion of bone marrow-derived progenitor cells and indirect mobilization of hematopoietic progenitor cells on atherosclerotic plaque and inflammatory process in atherosclerosis. Int J Cardiol, 2013. 168(5): p. 4769-74.
158. Balbarini, A., et al., Circulating endothelial progenitor cells characterization, function and relationship with cardiovascular risk factors. Curr Pharm Des, 2007. 13(16): p.
159. Xiao, Q., et al., Endothelial progenitor cells, cardiovascular risk factors, cytokine levels and atherosclerosis—results from a large population-based study. PLoS One, 2007. 2(10): p. e975.
160. Adams, B., Q. Xiao, and Q. Xu, Vascular progenitor cells and atherosclerosis. Future Cardiol, 2007. 3(6): p. 635-45.
161. Zenovich, A. G. and D. A. Taylor, Atherosclerosis as a disease of failed endogenous repair. Front Biosci, 2008. 13: p. 3621-36.
162. Briasoulis, A., et al., The role of endothelial progenitor cells in vascular repair after arterial injury and atherosclerotic plaque development. Cardiovasc Ther, 2011. 29(2): p. 125-39.
163. Steiner, S., et al., Endurance training increases the number of endothelial progenitor cells in patients with cardiovascular risk and coronary artery disease. Atherosclerosis, 2005. 181(2): p. 305-10.
164. Tousoulis, D., et al., Role of inflammation and oxidative stress in endothelial progenitor cell function and mobilization: therapeutic implications for cardiovascular diseases. Atherosclerosis, 2008. 201(2): p. 236-47.
165. Van Craenenbroeck, E. M. and V. M. Conraads, Endothelial progenitor cells in vascular health: focus on lifestyle. Microvasc Res, 2010. 79(3): p. 184-92.
166. Kawabe-Yako, R., et al., Cilostazol activates function of bone marrow-derived endothelial progenitor cell for re-endothelialization in a carotid balloon injury model. PLoS One, 2011. 6(9): p. e24646.
167. Kinoshita, M., et al., Long-term clinical outcome after intramuscular transplantation of granulocyte colony stimulating factor-mobilized CD34 positive cells in patients with critical limb ischemia. Atherosclerosis, 2012. 224(2): p. 440-5.
168. Oikonomou, E., et al., Atorvastatin treatment improves endothelial function through endothelial progenitor cells mobilization in ischemic heart failure patients. Atherosclerosis, 2015. 238(2): p. 159-64.
169. Caplice, N. M. and B. Doyle, Vascular progenitor cells: origin and mechanisms of mobilization, differentiation, integration, and vasculogenesis. Stem Cells Dev, 2005. 14(2): p. 122-39.
170. Nevskaya, T., et al., Circulating endothelial progenitor cells in systemic sclerosis: relation to impaired angiogenesis and cardiovascular manifestations. Clin Exp Rheumatol, 2008. 26(3): p. 421-9.
171. Hristov, M. and C. Weber, Endothelial progenitor cells in vascular repair and remodeling. Pharmacol Res, 2008. 58(2): p. 148-51.
172. Hristov, M. and C. Weber, Progenitor cell trafficking in the vascular wall. J Thromb Haemost, 2009. 7 Suppl 1: p. 31-4.
173. Bianconi, V., et al., Autologous Cell Therapy for Vascular Regeneration: The Role of Proangiogenic Cells. Curr Med Chem, 2018. 25(35): p. 4518-4534.
174. Ohtake, T., et al., Autologous Granulocyte Colony-Stimulating Factor-Mobilized Peripheral Blood CD34 Positive Cell Transplantation for Hemodialysis Patients with Critical Limb Ischemia: A Prospective Phase II Clinical Trial. Stem Cells Transl Med, 2018. 7(11): p. 774-782.
175. Zhu, J., et al., Single Intraosseous Simvastatin Application Induces Endothelial Progenitor Cell Mobilization and Therapeutic Angiogenesis in a Diabetic Hindlimb Ischemia Rat Model. Plast Reconstr Surg, 2021. 148(6): p. 936e-945e.
176. Tonnesen, M. G., X. Feng, and R. A. Clark, Angiogenesis in wound healing. J Investig Dermatol Symp Proc, 2000. 5(1): p. 40-6.

1. A method of treating viral induced sexual dysfunction comprising administering into cavernosal or clitoral tissue a regenerative cell population, or derivative of said regenerative population capable of stimulating one or more of the following: a) reduction of endothelial and/or smooth muscle apoptosis and/or neural cell apoptosis; b) stimulation angiogenesis; c) stimulation proliferation of smooth muscle; d) reducing fibrosis; e) augmentation of neurogenesis and f) suppression of inflammation.

2. The method of claim 1, wherein said derivative of said regenerative cell population is selected from a group comprising of: a) conditioned media; b) microvesicle; c) exosomes.

3. The method of claim 1, wherein said regenerative cell population is a mesenchymal or other regenerative cell population that is either autologous, allogeneic, or xenogeneic to the patient.

4. The method of claim 1, wherein said sexual dysfunction is caused by one or more of the following: a) a single stranded RNA virus; b) a double stranded RNA virus; c) a DNA virus; d) a coronavirus; and e) COVID-19.

5. The method of claim 1, wherein said sexual dysfunction is caused by inflammation associated with viral infection.

6. The method of claim 5, wherein said inflammation is associated with macrophage activation.

7. The method of claim 6, wherein said macrophage activation is associated with enhanced production of nitric oxide, and/or TNF-alpha, and/or interferon, and/or interleukin-33 as compared to a macrophage in a resting state.

8. The method of claim 5, wherein said inflammation is associated with increased activation of NF-kappa B in cells comprising caveronsal tissue.

9. The method of claim 8, wherein said cells comprising said cavernosal tissue are endothelial cells.

10. The method of claim 8, wherein said cells comprising said cavernosal tissue are monocytes.

11. The method of claim 8, wherein said cells comprising said cavernosal tissue are stem cells.

12. The method of claim 8, wherein said cells comprising said cavernosal tissue are smooth muscle cells.

13. The method of claim 38, wherein said cells comprising said cavernosal tissue are epithelial cells.

14. The method of claim 38, wherein said cells comprising said cavernosal tissue are telocytes.

15. The method of claim 38, wherein said cells comprising said cavernosal tissue are neurons.

16. The method of claim 38, wherein said cells comprising said cavernosal tissue are schwann cells.

17. The method of claim 15, wherein said inflammation is associated with increased activation of NF-kappa B in cells comprising clitoral tissue.

18. The method of claim 17, wherein said cells comprising said clitoral tissue are endothelial cells.

19. The method of claim 17, wherein said cells comprising said clitoral tissue are monocytes.

20. The method of claim 3, wherein said tissue derived mesenchymal stem cells are isolated from tissues containing cells selected from a group of cells comprising of: endothelial cells, epithelial cells, dermal cells, endodermal cells, mesodermal cells, stems, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue stems, corneal keratocytes, tendon stems, bone marrow reticular tissue stems, nonepithelial stems, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.
